(19) United States
(12) Plant Patent Application Publication
(10) Pub. No.: US 2007/0050874 P1
(43) Pub. Date: Mar. 1, 2007

(54) Helianthus plant named 'TABLE MOUNTAIN'

(76) Inventor: Keith Hammett, Massey, Auckland (NZ)

Correspondence Address: PLANT HAVEN, INC., P.O. BOX 3056, SANTA BARBARA, CA, 93130, US

(21) Appl. No.: 11/508,511

(22) Filed: Aug. 22, 2006

(30) Foreign Application Priority Data
Aug. 25, 2005 (NZ) SNF 004

(51) Int. Cl. A01H 5/00

(52) U.S. Cl. PLT/263

(57) ABSTRACT

A new cultivar of Helianthus named ‘TABLE MOUNTAIN’ that is distinguished by flat-topped compact habit, rich-green foliage, and deep-yellow daisy-like flowers in late summer. In combination these traits set ‘TABLE MOUNTAIN’ apart from all other existing varieties of Helianthus known to the inventor.

Genus: HELIANTHUS

Species: salicifolius

Denomination: ‘TABLE MOUNTAIN’

### BACKGROUND OF THE INVENTION

The present invention relates to a new and distinct cultivar of sunflower, a perennial herb that is grown for use as a specimen plant for borders. The new invention is known botanically as Helianthus salicifolius and will be referred to hereinafter by the cultivar name ‘TABLE MOUNTAIN’. Helianthus is in the family Compositae. The inflorescence is made up of a central cluster of disk florets surrounded by ray flowers.

‘TABLE MOUNTAIN’ is a hybrid seedling that resulted from a formal breeding program in Auckland, New Zealand. The breeding program was conducted by the inventor and began in 1990. ‘TABLE MOUNTAIN’ is the result of deliberate controlled cross-pollination using Helianthus ‘Golden Pyramid’ (unpatented) as the female parent and Helianthus ‘Autumn Queen’ (unpatented) as the male parent. With the intention of making a seedling selection, the parents were planted contiguously in isolation from other Helianthus, and insects permitted to effect pollination. The inventor selected ‘TABLE MOUNTAIN’ in 1993 based on the criteria of plant height and flat-topped compact habit.

The unique trait that distinguishes ‘TABLE MOUNTAIN’ from other varieties of the same genus is habit. ‘TABLE MOUNTAIN’ is distinguishable from the parents by shorter stature and flat-topped compact habit. The female parent ‘Golden Pyramid’ is 700-1000 mm in height and the male parent ‘Autumn Queen’ is 2 m in height. At maturity the new Helianthus variety named ‘TABLE MOUNTAIN’ reaches 400 mm. in height and 400 mm. in width.

‘TABLE MOUNTAIN’ exhibits flat-topped compact habit, rich-green foliage, and deep-yellow daisy-like blossoms. ‘TABLE MOUNTAIN’ reaches 400 mm. in height and 400 mm. in width at maturity, and is hardy to USDA Zone 3. The growing requirements are similar to the species. ‘TABLE MOUNTAIN’ is generally pest and disease free, however it can develop mildew in areas of high humidity, and it is susceptible to aphids. ‘TABLE MOUNTAIN’ is asexually propagated by summer tip-cuttings, and tissue culture. Cultural conditions include full sun, regular water and well-draining soil.

The first asexual reproduction of ‘TABLE MOUNTAIN’ was carried out in 2004 by the inventor in a cultivated area of Auckland, New Zealand. The method of asexual propagation used was tissue culture. Since that time ‘TABLE MOUNTAIN’ has proved fixed stable and reproduces true to type in subsequent generations of asexual propagation.

### SUMMARY OF THE INVENTION

The following traits have been repeatedly observed and represent the distinguishing characteristics of the new Helianthus cultivar. These traits in combination distinguish ‘TABLE MOUNTAIN’ from all other existing varieties of Helianthus known to the inventor. ‘TABLE MOUNTAIN’ has not been tested under all possible conditions and phenotypic differences may be observed with variations in environmental, climatic, and cultural conditions, however, without any variance in genotype.
1. ‘TABLE MOUNTAIN’ exhibits flat-topped compact habit.
2. ‘TABLE MOUNTAIN’ is a perennial herb used as a specimen plant for borders.
3. ‘TABLE MOUNTAIN’ exhibits deep-yellow daisy-like blossoms.
4. ‘TABLE MOUNTAIN’ grows in full sun and well-draining soil, with regular water.
5. ‘TABLE MOUNTAIN’ blooms in late summer.
6. ‘TABLE MOUNTAIN’ exhibits rich-green foliage.
7. ‘TABLE MOUNTAIN’ reaches 400 mm in height and 400 mm in width at maturity.
8. ‘TABLE MOUNTAIN’ is hardy to USDA Zone 3.

### BRIEF DESCRIPTION OF THE DRAWING

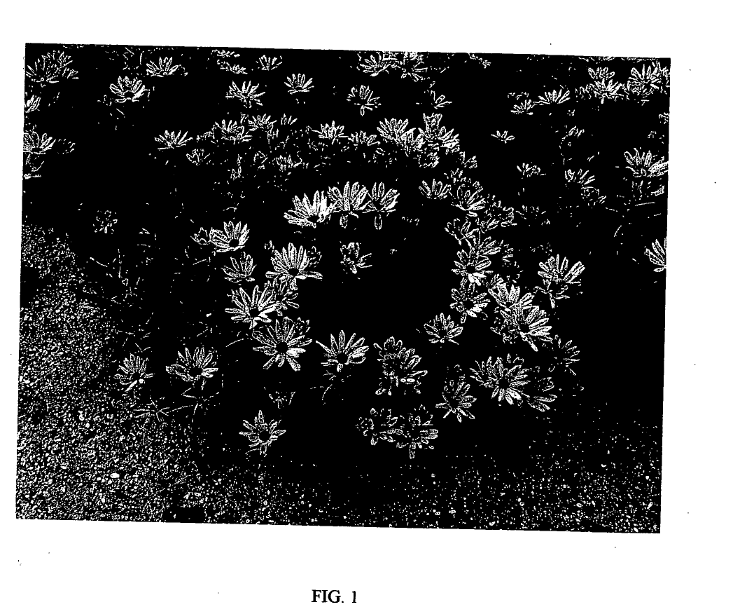

The accompanying color drawing labeled FIG. 1 illustrates the overall appearance of the new Helianthus cultivar named ‘TABLE MOUNTAIN’ showing the color as true as is reasonably possible to obtain in color reproductions of this type. Color in the photograph may differ from the color values cited in the detailed botanical description, which accurately describe the actual color of the new variety ‘TABLE MOUNTAIN’.

The drawing labeled FIG. 1 depicts habit and full bloom. FIG. 1 was made using conventional techniques and although flower and foliage color may appear different from actual color due to light reflectance they are as accurate as possible by conventional photography.

### BOTANICAL DESCRIPTION OF THE PLANT

The following is a detailed botanical description of the new Helianthus cultivar named ‘TABLE MOUNTAIN’. Observations, measurements, values and comparisons were collected in Arroyo Grande Calif. from 2-litre container plants. Color determinations are made in accordance with The 2001 Royal Horticultural Society Colour Chart from London England, except where general color terms of ordinary dictionary significance are used. Growing requirements are similar to the species.
Botanical classification: Helianthus salicifolius ‘TABLE MOUNTAIN’.
Family: Compositae.
Genus: HELIANTHUS.
Species: salicifolius.
Denomination: ‘TABLE MOUNTAIN’.
Common name: Sunflower.
Parentage: Helianthus salicifolius ‘TABLE MOUNTAIN’ is a hybrid seedling that resulted from deliberate controlled cross-pollination of the following parents:
   Female parent.—Helianthus ‘Golden pyramid’.
   Male parent.—Helianthus ‘Autumn Queen’.
Propagation method: Summer tip-cuttings, and tissue culture.
Rooting system: Fine and fibrous.
Vigor: Vigorous.
Time to develop roots: 14 to 20 days for cuttings to root.
Crop time: 10 weeks to 2 months to produce a finished commercial container plant.
Habit: Flat-topped compact habit.
Suggested container size: 2-litre container.
Use: Specimen plant for borders.
Type: Herbaceous perennial.
Dimensions at maturity: 400 mm in height and 400 mm in width.
Cultural requirements: Grow in full sun and well-draining soil with regular water.
Pest and disease: Generally pest and disease free, however can develop mildew in areas of high humidity, and is susceptible to aphids.
Hardiness: USDA Zone 3.
Seasonal interest: Flowers in August and September.
Stem:
   Branching habit.—Basal branching.
   Stem color.—146D.
   Stem dimensions.—34 cm in length and 0.50 cm in diameter.
   Stem shape.—Cylindrical.
   Stem surface.—Pubescent with long silky hairs.
   Internode length.—0.50 cm to 1 cm.
Foliage:
   Foliage type.—Evergreen.
   Leaf arrangement.—Alternate.
   Leaf division.—Simple.
   Leaf margin.—Barbellate.
   Leaf shape.—Narrow linear.
   Leaf length.—8 cm to 9.50 cm.
   Leaf width.—0.75 cm.
   Leaf base.—Truncate.
   Leaf apex.—Narrow acute.
   Leaf venation.—Prominent mid-vein.
   Vein color (abaxial and adaxial surfaces).—146D.
   Leaf surfaces (abaxial surfaces).—Pubescent.
   Leaf surfaces (adaxial surfaces).—Muriculate.
   Leaf attachment.—Sessile.
   Presence of stipules or spines.—None observed.
   Leaf color (adaxial surface).—139A and 148D.
   Leaf color (abaxial surface).—N138C.
   Fragrance.—Slight pine-like resinous scent.
Inflorescence:
   Inflorescence type.—Head.
   Aspect.—Facing upward.
   Inflorescence diameter.—70 mm.
   Disk diameter.—14 mm.
   Inflorescence quantity.—4-8 per lateral stem.
   Inflorescence shape.—Capitate.
   Bloom season.—Late summer.
   Peduncle dimensions.—4 cm in length and 0.25 cm in diameter.
   Peduncle shape.—Cylindrical.
   Peduncle surface.—Puberulent.
   Peduncle color.—148D and N187B.
   Bud color.—148D.
   Bud shape.—Flattened sphere.
   Bud surface.—Stipitate glandular.
   Bud apex.—Flattened to convex.
   Bud dimensions.—1 cm in height and 1 cm in width.
   Ray flower shape.—Elongated oblong.
   Ray flower quantity.—12 in number.
   Ray flower apex.—Retuse.
   Ray flower arrangement.—Radiate.
   Ray flower color (adaxial and abaxial surface).—14A.
   Ray flower length.—1.50 cm.
   Ray flower width.—0.20 cm.
   Ray flower form.—Petaloid.
   Ray flower surface.—Puberulent.
   Self-cleaning or persistent.—Self-cleaning.
   Disk floret shape.—Tubular.
   Disk floret color.—147A and 200A.
   Disk floret quantity.—75+ in number.
   Phyllaries (quantity per inflorescence).—25 in number.
   Phyllary length.—1 cm in length.
   Phyllary arrangement.—Imbricate involucre.
   Phyllary margin.—Entire.
   Phyllary form.—Sinuous.
   Phyllary apex.—Narrowly acute.
   Phyllary surface (adaxial and abaxial surfaces).—Puberulent.
   Phyllary shape.—Narrowly lanceolate.
   Phyllary color (adaxial and abaxial surfaces).—148D.
   Fragrance of inflorescence.—Slightly resinous.
Reproductive organs:
   Androecium.—Present on disk florets only.
   Anther color.—15A.
   Pollen.—Scarce.
   Pollen color.—21A.
   Gynoecium.—Present on both ray and disk florets only.
Seed: None observed to date.

1. A new and distinct cultivar of Helianthus plant named ‘TABLE MOUNTAIN’ as described and illustrated herein.
